(19) United States
(12) Patent Application Publication
(10) Pub. No.: US 2011/0166581 A1
(43) Pub. Date: Jul. 7, 2011

(54) SURGICAL DRILL AIMER

(76) Inventors: Willem Maré Van Der Merwe, Camps Bay (ZA); David Alexander James Wilson, Halifax (CA)

(21) Appl. No.: 13/063,118

(22) PCT Filed: Sep. 9, 2009
(86) PCT No.: PCT/IB2009/006790

§ 371 Date: Mar. 9, 2011

(30) Foreign Application Priority Data
Sep. 10, 2008 (ZA) 2008/07781

(51) Int. Cl. A61B 17/56

(52) U.S. Cl. 606/96

(57) ABSTRACT

A surgical drill aimer (1, 70, 100) is provided that is configured to be secured about a bone with a drill guide (8, 82, 108) on one side and a drill target (4, 88, 114) on an opposite side. A suture passage (40, 92, 120) is provided which is shaped to receive a suture (44, 94, 130) and has an exit (46, 90, 12) at or near the drill target and configured such that a suture extending therefrom is accessible through a drill hole formed using the drill aimer and opening in line with the drill target.

### FIELD OF THE INVENTION

This invention relates to a surgical drill aimer, more particularly, but not exclusively, to a drill aimer for use in arthroscopic surgery.

### BACKGROUND TO THE INVENTION

Arthroscopic surgery is often the preferred surgical procedure for the treatment of many different injuries such as knee injuries, an example of which is the reconstruction of the posterior cruciate ligament (PCL). During arthroscopic surgery such as PCL reconstructive surgery, a drill aimer, also referred to as a drill guide or drill jig, is often used to facilitate precise formation of a drill hole through a bone. These devices may include a pair of arms or jaws, each operatively located on opposite sides of the bone. One arm carries a guide, typically a sleeve or tube, for the drill and the end of the other carries a bone contacting surface. This contacting surface acts as the target for the drill and typically has a central aperture through which the drill tip projects on exiting the bone. Numerous devices exist and some are described in the following patents: U.S. Pat. No. 4,257,411, U.S. Pat. No. 4,672,957, U.S. Pat. No. 5,154,720, U.S. Pat. No. 5,350,383, EP 0797955 and U.S. Pat. No. 6,918,916.

In this specification, a “drill guide” has its widest meaning and includes any device or formation shaped to receive a drill or drill bit and guide it along a drill path towards a drill target. Similarly, “drill target” has its widest meaning and includes any device or formation which denotes the end of a drill path and which typically abuts a bone on the opposite side to the drill guide.

Performing PCL reconstructive surgery arthroscopically requires specialised skills as it is greatly complicated by the poor visibility at the tibial insertion of the PCL. A suture must be passed through this point but this is very difficult to achieve as the tip of the suture cannot be visualized due to the poor visibility caused by the surrounding soft tissue. Further complicating the situation is the presence of the poplitial neurovascular bundle which lies directly posterior to the tibial insertion of the PCL. Because of the location of these sensitive tissues the surgeon cannot debride aggressively and must be very careful with any instrumentation.

One commonly used technique for alleviating this problem of visualizing the insertion point of the PCL is to open a posterior portal. However, this both adds approximately 30 minutes on to the surgery and increases both operative and post-operative complications. Most surgeons thus try and avoid this technique.

As an alternative, a device is available which attempts to place the suture in position. This device, known as The Worm', and manufactured by Smith and Nephew, includes a pre-curved nitonal wire with a suture attached to it and constrained by a straight metal tube. The metal tube with the wire and suture inside it are passed up the trans-tibial drill hole, formed using a drill guide, and once the metal tube has entered the joint capsule the nitonal wire is extended past the tip of the tube and springs into its preformed shape, ideally presenting an easily visualized suture. Unfortunately, the wire rarely springs up in the expected direction and frequently many attempts are necessary to adequately visualize the suture. Even highly experienced surgeons often require 20 minutes or more to capture and pull the suture through the trans-tibial drill hole.

### OBJECT OF THE INVENTION

It is an object of this invention to provide a surgical drill aimer which will at least partially alleviate some of the abovementioned problems.

### SUMMARY OF THE INVENTION

In accordance with this invention there is provided a surgical drill aimer configured to be secured about a bone with a drill guide on one side and a drill target on an opposite side, characterised in that a suture passage is provided which is shaped to receive a suture and has an exit at or near the drill target and configured such that a suture extending therefrom is accessible through a drill hole opening in line with the drill target.

Further features of the invention provide for the entrance to the suture passage to be remote from the exit; for the entrance to be accessible from a convenient location, preferably externally of a joint capsule; for the suture passage to be defined by a tube secured to the drill aimer in which instance the tube may optionally be releasably secured to the drill aimer; and, in the alternative, for the suture passage to be formed in an arm or element of the drill aimer on which the drill target is carried.

Still further features of the invention provide for the drill target to include an opening shaped to receive the tip of a drill bit and for the exit of the suture passage to open into the opening; and for the exit to be configured such that a suture extending therefrom is directed into a drill hole opening onto the drill target.

Yet further features of the invention provide for a suture passage to be configured to releasably secure a suture adjacent the drill target; and for the suture passage to include a pair of surfaces biased towards each other between which a suture can be releasably secured.

In order that the invention may be more fully understood different embodiments thereof will now be described with reference to the accompanying drawings.

### BRIEF DESCRIPTION OF THE DRAWINGS

In the drawings:

FIG. 1 is plan view of a first embodiment of a drill aimer;

FIG. 2 is a perspective view of the drill aimer in FIG. 1 in use;

FIG. 3 is a plan view of a second embodiment of a drill aimer; and,

FIG. 4 is a plan view of a third embodiment of a drill aimer.

### DETAILED DESCRIPTION WITH REFERENCE TO THE DRAWINGS

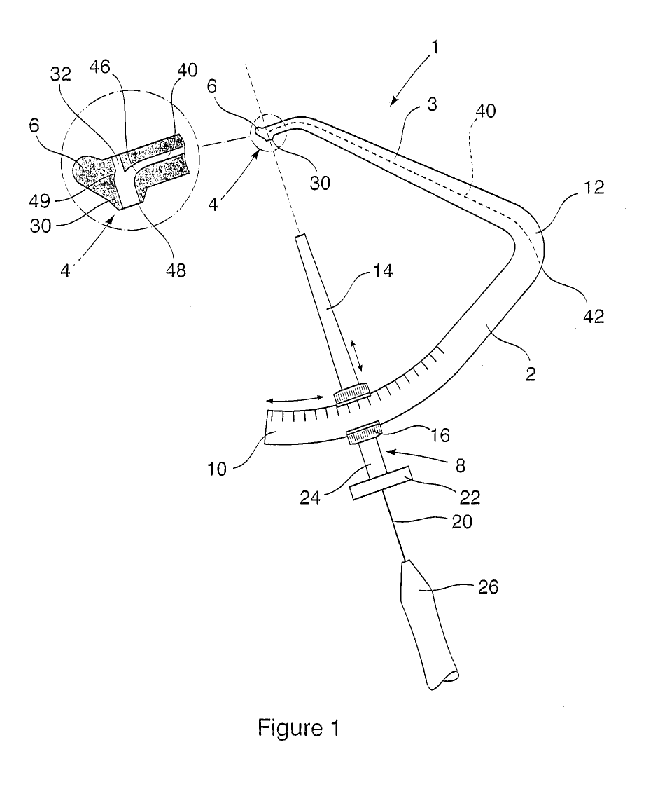

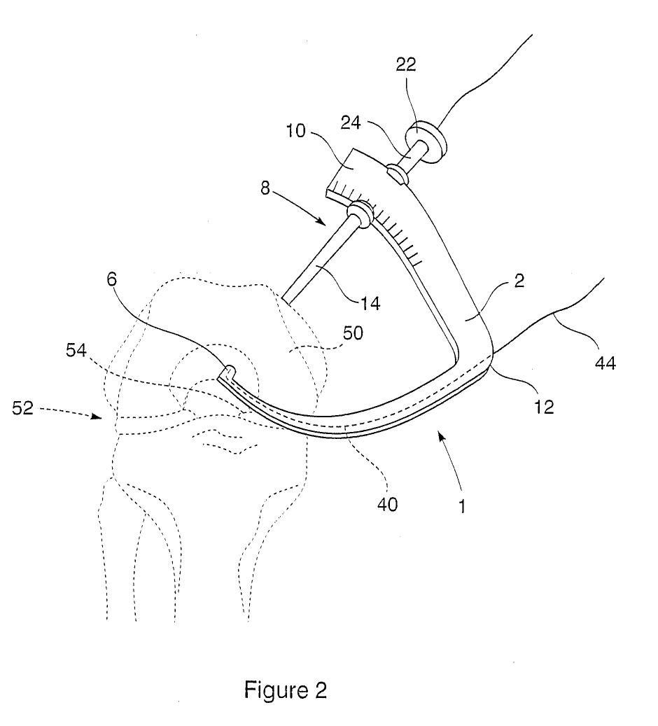

In the embodiment of the invention illustrated in FIGS. 1 and 2, a drill aimer (1) includes a first arm (2) and a second arm (3) joined at one end to form a generally V-shaped arm assembly. The second arm (3) has a drill target (4) at its free end (6) and the first arm carries a drill guide (8) towards its free end (10). The second arm (3) tapers from the bend or junction (12) towards the drill target (4) and is also slightly curved towards the free end (6). The first arm (2), on the other hand has a substantially constant width from the apex (12) towards its free end (10).

The drill guide (8) includes an elongate drill sleeve (14) which is slideably secured in a carriage (16) which is in turn adjustably secured to the first arm (2) in a region thereof that is arcuate so that the axis of the drill sleeve remains directed towards the drill target through a range of different positions. The sleeve (14) is shaped to receive a drill bit (20) therethrough and has a flange (22) at its end (24) distal from the drill target (6) which both allows it to be easily gripped and manipulated and also acts as a stop for a drill (26).

The drill target (4) includes a cylindrical projection (30) on the second arm (3) with an axially extending aperture (32) therethrough directed towards the carriage (16) on the first arm (2). Thus far, the drill aimer (1) has features which are known in the art in that a drill target and drill guide are provided which are adjustable relative to each other and enable a bone to be secured therebetween.

According to the invention, a suture passage (40) is provided in the second arm (2) extending generally from the apex (12) to the drill target (6). In this embodiment, the entrance (42) of the passage (40) opens in generally the same direction of that of the sleeve (14) and is chamfered to facilitate insertion of a suture (44) therein. The exit (46) of the passage (40) opens into the aperture (32) in the drill aimer (4) and is slightly curved towards the drill guide end (48) thereof. The wall of the aperture (32) opposite the exit (46) of the passage (40) is radially inwardly inclined from the drill guide end (48). The reason for this will become apparent from the further description below.

As shown in FIG. 2, in use the drill aimer (1) is secured about a bone, in this embodiment a femoral condyle (50) of a knee joint (52) with the drill target (6) located within the anteromedial portal (54). Once a hole or tunnel (not shown) has been drilled through the bone the drill is removed and a suture (44) then fed into the passage (40) through the entrance (42) which is easily accessible to the surgeon. At the exit (46) to the passage (40) the tip of the suture (44) is directed towards the drill guide (8) by both the curvature of the passage (40) and the inclined side (49) of the aperture (32) which it strikes after exiting the passage (40). Feeding the suture (44) further into the passage (40) causes the end to enter the tunnel in the bone and thereafter the drill sleeve (14) which it eventually exits. Hereafter, it is a simple matter to draw the suture (44) through the tunnel and thereafter proceed in the normal fashion with the operation. This may include attaching a Gortex™ rasper to an end of the suture and pulling it back through the tunnel.

Groups The provision of the passage according to the invention can be fairly easily achieved with most or all drill aimers and results in a considerabe time saving to the surgeon. It also enhances patient safety as it eliminates the need for a posterior inspection port or for debridement of the internal tissue with its concomitant risk of injury to nerves, arteries or veins.

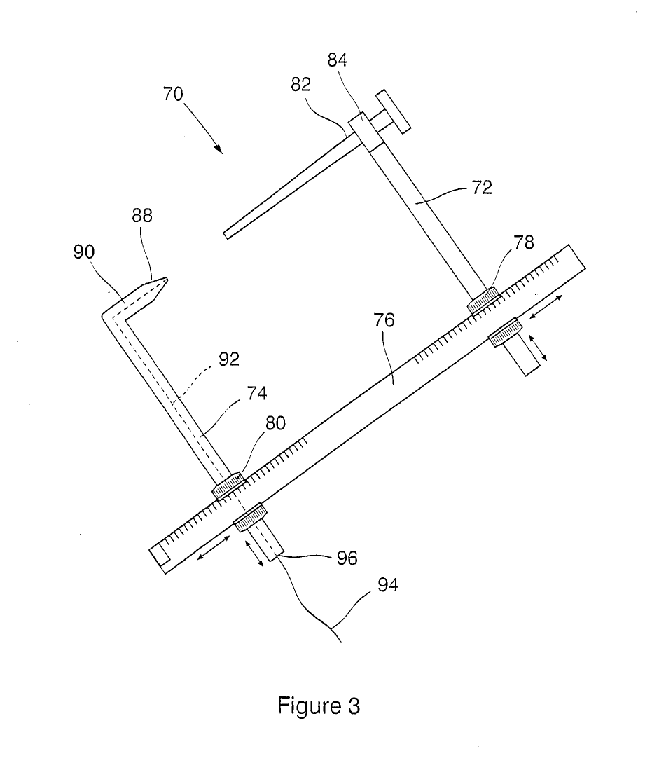

It will be appreciated that the drill aimer can have any suitable configuration. As shown in FIG. 3, the drill aimer (70) could have first and second arms (72, 74) adjustably secured to an elongate bar (76) through carriages (78, 80) to which they are themselves adjustably secured. In this embodiment, the first arm (72) carries a drill guide (82) at its end (84) distal from the bar (76) whilst the second arm (74) terminates in a drill target (88) which is directed towards the drill guide (82) and has an aperture (90) extending therethrough. In this embodiment, the passage (92) for the suture (94) extends centrally through the second arm (74) between the aperture (90) and the opposite end (96) of the arm (92). The bar (76) could be arcuate and either of the arms could be fixed relative thereto.

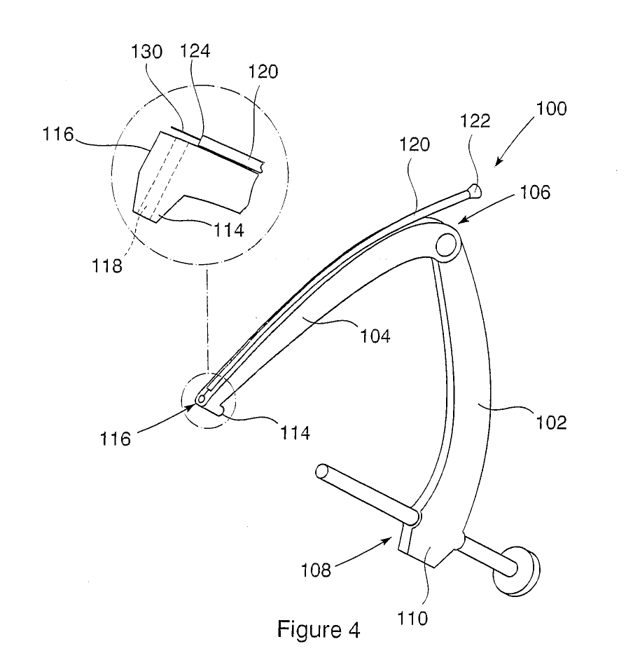

Alternatively, as shown in FIG. 4, the drill aimer (100) could have forst and second arms (102, 104) hingedly secured together at one end (106) to form a V-shape. A drill guide (108), in this embodiment an elongate sleeve similar to that described with reference to FIG. 1, is adjustably secured adjacent the end (110) of the first arm (102) with the drill target (114) provided at the free end (116) of the second arm (104) with an aperture (118) extending therethrough generally aligned with the drill sleeve (108).

In this embodiment, the suture passage is defined by a tube (120) which extends slightly past the hinge (106) and has a flared entrance (122). The opposite end (124) or exit of the tube (120) is adjacent the aperture (118) on the end opposite the drill guide (108). A suture (130) fed through the passage defined by the tube (120) projects from the exit (124) over the aperture (118) where it can be retrieved by a suitable tool (not shown).

The tube (120) could be removably secured to the drill aimer (100) if desired and could be made of any suitable material. Although stainless steel is preferred, a plastics material could also be used to permit the tube to be readily disposable.

It will be appreciated that the passage can have any suitable configuration provided that it either permits a suture to be fed into the drill hole and then into the drill guide or positions the suture adjacent the drill hole or drill target so that it can be retrieved using a tool inserted into the drill hole, typically through the drill guide. The passage need not be enclosed but could also be partially open or channel shaped.

Typically, the entrance to the suture passage is remote from the exit and positioned so as to be conveniently accessible to a surgeon. Clearly the exact location of the entrance will be to a large extent dependant on the configuration of the drill aimer. However, if desired, the suture passage could be provided by a notch or similar formation which merely holds the suture in position adjacent the drill target during positioning and drilling and from which the suture can easily be retrieved once the drill hole has been formed.

It will still further be appreciated that the suture passage can be configured to receive any suitable material and that it is not necessary that only a suture be fed therethrough.

The drill aimer can have any suitable configuration and can include means for stabilising or locking the device in position. Also, the drill target need not have an aperture therein, and could, for example, be a C-shaped. In such an event the passage will be suitably configured to allow the suture to be fed into a position where it can be easily retrieved.

1. A surgical drill aimer configured to be secured about a bone with a drill guide (8, 82, 108) on one side and a drill target on an opposite side, charactcriccd in that wherein a suture passage is provided which is shaped to receive a suture and has an exit at or near the drill target and configured such that a suture extending therefrom is accessible through a drill hole opening in line with the drill target.

2. A surgical drill aimer as claimed in claim 1 in which the entrance to the suture passage is operatively external of a joint capsule with the drill aimer secured about a bone joint.

3. A surgical drill aimer as claimed in claim 1 in which the entrance to the suture passage is remote from the exit.

4. A surgical drill aimer as claimed in claim 1 in which the suture passage is defined by a tube secured to the drill aimer.

5. A surgical drill aimer as claimed in claim 4 in which the tube is releasably secured to the drill aimer.

6. A surgical drill aimer as claimed in claim 1 in which the suture passage is formed in an arm or element of the drill aimer on which the drill target is carried.

7. A surgical drill aimer as claimed in claim 1 in which the drill target includes an opening shaped to receive the tip of a drill bit.

8. A surgical drill aimer as claimed in claim 1 in which the exit of the suture passage opens into the opening.

9. A surgical drill aimer as claimed in claim 1 in which the exit is configured such that a suture extending therefrom is directed into a drill hole opening onto the drill target.

10. A surgical drill aimer as claimed in claim 1 in which the suture passage is configured to releasably secure a suture adjacent the drill target.
